(19) United States
(12) Patent Application Publication
(10) Pub. No.: US 2007/0193578 A1
(43) Pub. Date: Aug. 23, 2007

(54) Combination Pressure Therapy for Treatment of Ischemia & Heart Conditions, Diabetes, Alzheimer's Disease and Cancer

(75) Inventors: Carl Linton, Temecula, CA (US); Allen Ruszkowski, Temecula, CA (US); Thomas Jackson Tidwell, Columbus, GA (US)

Correspondence Address: WILSON SONSINI GOODRICH & ROSATI, 650 PAGE MILL ROAD, PALO ALTO, CA, 94304-1050, US

(73) Assignee: CVAC SYSTEMS, INC., Temecula, CA (US)

(21) Appl. No.: 11/672,934

(22) Filed: Feb. 8, 2007

Related U.S. Application Data
(60) Provisional application No. 60/771,848, filed on Feb. 8, 2006.
(60) Provisional application No. 60/772,647, filed on Feb. 10, 2006.
(60) Provisional application No. 60/773,460, filed on Feb. 15, 2006.
(60) Provisional application No. 60/773,585, filed on Feb. 15, 2006.
(60) Provisional application No. 60/774,441, filed on Feb. 17, 2006.
(60) Provisional application No. 60/775,917, filed on Feb. 22, 2006.
(60) Provisional application No. 60/775,521, filed on Feb. 21, 2006.
(60) Provisional application No. 60/745,723, filed on Apr. 26, 2006.
(60) Provisional application No. 60/824,890, filed on Sep. 7, 2006.
(60) Provisional application No. 60/822,375, filed on Aug. 14, 2006.
(60) Provisional application No. 60/826,061, filed on Sep. 18, 2006.
(60) Provisional application No. 60/826,068, filed on Sep. 18, 2006.
(60) Provisional application No. 60/743,470, filed on Mar. 13, 2006.
(60) Provisional application No. 60/745,721, filed on Apr. 26, 2006.

(51) Int. Cl. A61K 38/28; A61M 16/00

(52) U.S. Cl. 128/200.24; 514/2; 514/3

(57) ABSTRACT

Methods for administering pressure changes to a user for the treatment and prevention of diseases and conditions are disclosed herein. Methods of administering Cyclic Variations in Altitude Conditioning Sessions (CVAC Session(s)) for the treatment of ischemia, diabetes and associated complications, Alzheimer's disease, and cancer are disclosed herein.

### CROSS-REFERENCE

This application claims the benefit of U.S. Provisional Application No. 60/771,848, filed Feb. 8, 2006, U.S. Provisional Application No. 60/772,647, filed Feb. 10, 2006, U.S. Provisional Application No. 60/773,460, filed Feb. 15, 2006, U.S. Provisional Application No. 60/773,585, filed Feb. 15, 2006, U.S. Provisional Application No. 60/774,441, filed Feb. 17, 2006, U.S. Provisional Application No. 60/775,917, filed Feb. 22, 2006, U.S. Provisional Application No. 60/775,521, filed Feb. 21, 2006, U.S. Provisional Application No. 60/743,470, filed Mar. 13, 2006, U.S. Provisional Application No. 60/745,721, filed Apr. 26, 2006, U.S. Provisional Application No. 60/745,723, filed Apr. 26, 2006, U.S. Provisional Application No. 60/824,890, filed Sep. 7, 2006, U.S. Provisional Application No. 60/822,375, filed Aug. 14, 2006, U.S. Provisional Application No. 60/826,061, filed Sep. 18, 2006, and U.S. Provisional Application No. 60/826,068, filed Sep. 18, 2006, which applications are incorporated herein by reference.

### FIELD OF THE INVENTION

The invention relates to the use of air pressure therapy for the treatment and prevention of diseases and conditions that benefit from hypoxic conditioning.

### BACKGROUND OF THE INVENTION

Tissues deprived of blood and oxygen suffer ischemic necrosis or infarction, often resulting in permanent tissue damage. Cardiac ischemia is often termed “angina”, “heart disease”, or a “heart attack”, and cerebral ischemia is often termed a “stroke”. Both cardiac and cerebral ischemia result from decreased blood and oxygen flow which is often followed by some degree of brain damage, damage to heart tissue, or both. The decrease in blood flow and oxygenation may be the result of occlusion of arteries, rupture of vessels, developmental malformation, altered viscosity or other quality of blood, or physical traumas. Prior to an actual heart attack, cardiac ischemia can present as angina pectoralis. Angina pectoralis is the moderate to severe pain experienced in the chest as a result of ischemia in the cardiac vessels and tissue. It is indicative of worsening blockage of the cardiac arteries, and typically precedes an ischemic event such as a heart attack. Furthermore, myocardial ischemia can result in a progressive disease termed congestive heart failure. Congestive heart failure is a condition where the heart can no longer efficiently pump sufficient volumes of blood to the body. This weakening of the heart often results from myocardial ischemia that stresses or damages the cardiac tissue. Congestive heart failure can also manifest following one or more heart attacks that have weakened the cardiac tissue or resulted in scar tissue build-up in the heart. Regardless of the mechanism of ischemia, the complication of congestive heart failure can be associated with or result from cardiac ischemia.

Type 2 Diabetes (i.e., diabetes mellitus, non-insulin dependent diabetes mellitus, adult onset diabetes) is frequently thought of as a disease caused by high blood sugar, medical research has moved towards an understanding of abnormal blood glucose levels as a symptom of an underlying disease related to dysregulated fat metabolism. Thus high fatty acid levels lead to a range of lipotoxicities such as insulin resistance, pancreatic beta cell apoptosis, and a disorder termed “metabolic syndrome.” Similarly, metabolic syndrome may involve dysregulated glucose transport which contributes to cellular resistance to insulin and is influenced by increased fatty acid levels in the blood. [Schulman, G., Cellular Mechanisms of Insulin Resistance, J. Clin. Invest., 106(2): 171-76 (2000).] Insulin resistance is typically detected by an increased level of blood insulin, increased blood levels of glucose in response to oral glucose tolerance test (OGTT), or decreased levels of phosphorylated protein kinase B (AKT) in response to insulin administration. Insulin resistance may be caused by decreased sensitivity of the insulin receptor-related signaling system in cells and/or by loss of beta cells in the pancreas. There is also evidence that insulin resistance can be characterized as having an underlying inflammatory component.

Sedentary lifestyle and obesity have contributed to the increased occurrence of Type 2 Diabetes. Therapeutic intervention has been aimed at people with impaired glucose tolerance (IGT). IGT is defined as hyperglycaemia (with glucose values intermediate between normal and diabetes) following a glucose load, and affects at least 200 million people worldwide. People afflicted with IGT possess a higher future risk than the general population for developing diabetes. Approximately 40% of people with IGT progress to diabetes in 5-10 years, but some revert to normal or remain IGT. Moreover, people with IGT also have a heightened risk of developing cardiovascular disease, such as hypertension, dyslipidaemia and central obesity. Thus, the diagnosis of IGT, particularly in apparently healthy and ambulatory individuals, has important prognostic implications. For a more detailed review, see Zimmet P, et al., Nature, 414:783-7 (2001), the disclosure of which is incorporated herein by reference. Recently, impaired fasting glucose (IFG) was introduced as another category of abnormal glucose metabolism. IFG is defined on the basis of fasting glucose concentration and, like IGT, it is also associated with risk of cardiovascular disease and future diabetes.

Type 2 Diabetes or abnormal glucose metabolism may be caused by a variety of factors and may manifest heterogeneous symptoms. Previously, Type 2 Diabetes was regarded as a relatively distinct disease entity, but current understanding has revealed that Type 2 Diabetes (and its associated hyperglycaemia or dysglycaemia) is often a manifestation of a much broader underlying disorder, which includes the metabolic syndrome as noted above. This syndrome is sometimes referred to as Syndrome X, and is a cluster of cardiovascular disease risk factors that, in addition to glucose intolerance, includes hyperinsulinaemia, dyslipidaemia, hypertension, visceral obesity, hypercoagulability, and microalbuminuria. Many complications can result from the symptoms of diabetes. Such complications include the metabolic syndromes detailed above as well as vision disorders, neuropathy, kidney disease, and vascular diseases such as heart disease, stroke, and extremity ulceration/amputation. The problems associated with diabetes are debilitating and often fatal, thus treatment of diabetes is paramount to prevention of these severe complications.

Recent understanding of the factors leading to Type 2 Diabetes has influenced contemporary therapy for the disease to the extent that more aggressive approaches to treating hyperglycaemia as well as other risk factors such as hypertension, dyslipidaemia and central obesity in type 2 diabetics have been pursued. Therapies for Type 2 Diabetes range from administration of pharmaceuticals to changes in lifestyle with insulin administration and dietary control being the primary therapies. However, insulin administration and monitoring requires the daily use of needles, and compliance with such regimens is often problematic. Similarly, dietary and exercise changes, while effective at improving glucose tolerance, often fail to become an integral part of a diabetic sufferer's life to the degree necessary to alleviate the levels of pharmaceuticals needed as well as the complications associated with the disease.

The usual first symptom noticed in Alzheimer's disease is memory loss which progresses from seemingly simple and often fluctuating forgetfulness to a more pervasive loss of recent memory, then of familiar and well-known skills or objects or persons. Aphasia, disorientation and disinhibition usually accompany the loss of memory. Alzheimer's disease may also include behavioral changes, such as outbursts of violence or excessive passivity in people who have no previous history of such behavior. In the later stages, deterioration of musculature and mobility, leading to bedfastness, inability to feed oneself, and incontinence, will be seen if death from some external cause (e.g. heart attack or pneumonia) does not intervene.

Additionally, the presence of cardiovascular risk factors—diabetes, hypertension, high cholesterol and smoking—in middle age (ages 40 to 44) was found very strongly associated with late-life dementia [Whitmer, R. A., et al., Midlife cardiovascular risk factors and risk of dementia in late life, Neurology, 64:277-281 (2005)]. Some studies have indicated that non-steroidal anti-inflammatory drugs (NSAIDs) like ibuprofen and aspirin may delay the onset, and lower the ultimate risk, of Alzheimer's disease. According to population studies, low but consistent daily NSAID used over a period of years such as ibuprofen (Advil®, Motrin®) seems to slow the progress of Alzheimer's. NSAIDs may affect the onset of the disease, but they are of little use for treating it once it has progressed to early or full-blown Alzheimer's. Additionally, the combination of vitamins such as E and C might, over time, sharply reduce the risk of Alzheimer's disease, but only if dosage is 400 i.u. per day of vitamin E plus 500 mg or more per day of vitamin C. Lesser amounts, such as those found in multivitamin pills, appeared markedly less effective. [Zandi, P. P., et al., Reduced risk of Alzheimer disease in users of antioxidant vitamin supplements: the Cache County Study, Arch. Neurol., 61:82-88 (2004).]

Cancerous cells, growths, and tumors also represent an on-going challenge for effective treatment with most chemotherapeutic drugs and agents, radiation therapy, and other methods. For example, as a tumor increases in size, it reduces or cuts off blood supply to the internal core of the tumor due to the increasing blood, nutrient, and oxygen needs of the outer, high growth area. This results in a hypoxic center of the tumor and selects for the growth of hypoxia-tolerant cells in the core. These core cells are more resistant to radiation as well as chemotherapeutic agents due to the lack of blood supply, nutrients, and a resultant lack of oxygen. This lack of blood and oxygen prevents chemotherapeutic agents and other compounds (antibodies, protein therapies, etc.) from entering the tumor core and reacting with oxygen to exert their therapeutic effects. Similarly, ionizing radiation therapy often fails due to the lack of reactive oxygen available for peroxide and radical formation within the hypoxic tumor core. Thus, when treatments of chemotherapy and/or radiation are administered to a patient with a cancerous tumor, the outer cells of the tumor are killed, but the cells of the internal core are not. The tumor, even with generally effective treatment, may continue to thrive and metastasize, necessitating additional therapy sessions and often higher dosages of chemotherapy, radiation, alternative compound therapies, or a combination thereof.

### SUMMARY OF THE INVENTION

The present invention provides for a method of administering pressure changes (CVAC) to a user for the prevention, treatment, and amelioration of ischemic disease and complications associated with or arising from such disease. Ischemic disease encompasses cerebral ischemia, strokes, ischemic heart disease, heart attacks, arteriosclerosis, atherosclerosis, congestive heart failure, and myriad associated cerebral and cardiac conditions associated with blockages of blood vessels, ruptures of vessels, loss in blood pressure, and damage to surrounding tissues. Application of the disclosed methodologies helps to prevent the onset of ischemic disease, treats asymptomatic and symptomatic disease, and aids in recovery from ischemic disease, ischemic events, complications associated with or arising from ischemia, and associated surgery. Similarly, the present invention also provides for a method of administering CVAC to a user for the treatment of diabetes, where treatment of diabetes includes prevention and/or amelioration of diabetes, metabolic syndrome (Syndrome X), and complications associated with diabetes. Furthermore, the present invention provides for a method of administering CVAC to a user for the treatment of Alzheimer's disease, including complications thereof. Again, treatment of Alzheimer's disease includes prevention, amelioration and treatment of the disease. Finally, the present invention provides for a method of administering CVAC to a user for the treatment of cancer. Treatment of cancer as defined herein includes prevention, amelioration, treatment, and aids to recovery from cancer therapies.

One aspect of the invention is the administration of one or more Cyclic Variations in Altitude Conditioning Sessions (CVAC sessions) for the treatment of ischemic disease. In an embodiment of the invention, at least one CVAC session is administered prior to the onset of ischemic disease, and CVAC sessions may be administered in defined intervals. In additional embodiments, CVAC sessions are administered following an ischemic event and/or prior to surgery related to ischemic disease. The effect of such administration is a lessening of ischemic symptoms, reduction in ischemic damage to tissues, and/or reducing the detrimental effects of ischemic events.

A CVAC session consists of a set of targets which are pressures found in the natural atmosphere. These targets are delivered in a precise order. The starting points and ending points in any CVAC Session are preferably the ambient pressure at the delivery site. The targets inherent in any CVAC Session are connected or joined together by clearly defined transitions. These transitions are either rises in pressure or falls in pressure, or a combination of the two. Additional targets which modulate time, temperature, or humidity are also run concurrently, sequentially, or at other intervals with the pressure targets when such additional targets and conditions are desired.

An embodiment of the invention is the administration of CVAC sessions for the treatment of cerebral ischemia and related ischemic events. Further embodiments of the invention include administering the CVAC sessions prior to cerebral ischemic events and subsequent to ischemic events to treat, prevent, or ameliorate the effects of cerebral ischemia. Even further embodiments include administration of CVAC sessions prior to and after surgeries related to cerebral ischemia for the prevention and amelioration of detrimental effects resulting from such surgeries.

A further embodiment of the invention is the administration of CVAC sessions for the treatment of ischemic heart disease and related ischemic events. Further embodiments of the invention include administering the CVAC sessions prior to and subsequent to cardiac ischemic events to treat, prevent, or ameliorate the effects of ischemic heart disease.

An additional embodiment of the invention is the administration of CVAC sessions for the treatment, prevention, and/or amelioration of congestive heart failure. Even further embodiments include administration of CVAC sessions prior to and after surgeries related to ischemic heart disease for the prevention and/or amelioration of detrimental effects resulting from such surgeries.

A second aspect of the present invention provides for a method of administering CVAC sessions to a user for the purpose of treating diabetes and/or complications associated therewith or resulting therefrom. One embodiment of the invention is the administration of CVAC sessions for the treatment of diabetes. In another embodiment of the invention, at least one CVAC session is administered to facilitate the treatment of diabetes. Another aspect of the invention is the administration of at least one CVAC session for the reduction of dependence upon traditional therapies for diabetes, e.g. pharmaceuticals such as insulin. A further aspect of the invention is the administration of CVAC sessions for the treatment of complications of diabetes. Yet an additional aspect of the invention is the administration of CVAC sessions for the treatment of metabolic syndrome.

A third aspect of the invention is the administration of CVAC sessions for the treatment of Alzheimer's disease. In an embodiment of the invention, at least one CVAC session is administered to prevent or slow the progression of Alzheimer's disease. In another embodiment, at least one CVAC session is administered to prevent Alzheimer's disease. CVAC sessions may be administered at defined intervals or at random occurrences. The effect of such administration is a lessening of amyloid deposits and/or neural degeneration as well as improved fluid exchange and/or drainage from the affected areas.

A fourth aspect of the invention is the administration of CVAC sessions for the treatment of cancer, cancerous tumors, or combinations thereof. In an embodiment of the invention, at least one CVAC session is administered prior to a treatment of cancer and/or in anticipation of surgery for cancer, or combinations thereof. A further embodiment includes administration of at least one CVAC session during a treatment for cancer. Multiple CVAC sessions may be administered in defined intervals or at random intervals. In additional embodiments, CVAC sessions are administered following a treatment for cancer and/or cancerous tumors. The effect of such administration is a slowing of the growth of the cancer, a reduction in the size of the cancerous tissue, preventing the metastasis of the cancer, or reducing the detrimental effects of known chemotherapies, radiation therapies, other known cancer therapies, and/or combinations thereof.

In additional embodiments, including the aforementioned aspects and embodiments, the targets of the CVAC sessions include pressure, temperature, time, and humidity parameters. Parameters of targets and sessions can be customized to individual needs. In still further embodiments of the invention, including the aforementioned aspects and embodiments, CVAC sessions are administered in combination with pharmaceutical regimens for the treatment, prevention, or amelioration the aforementioned conditions and diseases. Further embodiments, including the aforementioned embodiments and aspects, include administration of CVAC sessions in combination with alternative therapies and non-pharmaceutical therapies for the treatment of the aforementioned diseases, conditions, and syndromes as defined herein.

### BRIEF DESCRIPTION OF THE DRAWINGS

FIG. 1A depicts a graphed profile of the various pressures applied over time during an exemplary CVAC session. The Y-axis represents atmospheric pressure levels and the X-axis represents time. The varying pressures, as indicated by the changes in values on the Y-axis, were applied for various lengths of time, as indicated by changes values on the X-axis. The exemplary CVAC session depicted in FIG. 1A was 20 minutes in length.

FIG. 1B depicts a different graphed profile of the pressures applied over time during another exemplary CVAC session. The Y-axis again represents atmospheric pressure levels and the X-axis represents time. Different pressures were again applied, as indicated by changes in value on the Y-axis, for various lengths of time, as indicated by the changes in values on the X-axis. This exemplary CVAC session was also 20 minutes in length.

### DETAILED DESCRIPTION OF THE INVENTION

While oxygen deprivation of the body or specific tissues can cause tissue damage, and even death, controlled deprivation of oxygen to the body and/or specific tissues has been shown to be beneficial when imposed for specific periods of time under particular conditions. In practice, most current hypoxic conditioning protocols utilize static pressures for blocks of time ranging from 30 minutes to an hour or more to achieve the desired and reported responses. Hypoxic conditioning may be provided by decreased oxygen levels in the atmosphere or by a reduction in atmospheric pressure (hypobaric conditions), thus reducing the availability of oxygen for efficient respiration. Both methods can provide beneficial results.

Hypoxic Conditioning

Moderate static hypoxic preconditioning is known to provide protection from ischemic damage via tolerance. When the environmental oxygen levels are reduced (hypoxia), downstream effects include protection from damage due to subsequent hypoxia or ischemia. [Sharp, F., et al., Hypoxic Preconditioning Protects against Ischemic Brain Injury, NeuroRx: J. Am. Soc. Exp. Neuro., Vol. 1: 26-25 (2004)]. This tolerance is not yet completely understood, but it has been linked to various cellular mechanisms and molecules, including, but not limited to, molecules such as erythropoietin (EPO), hypoxia-inducible factor (HIF), Tumor Necrosis Factor (TNF), glycogen, lactate, and others. [Sharp, F., et al., Hypoxic Preconditioning Protects against Ischemic Brain Injury, NeuroRx: J. Am. Soc. Exp. Neuro., Vol. 1: 26-25 (2004)]. Additionally, beneficial static hypoxic conditioning is not purely additive. Administration of sequential sessions can have detrimental effects. Oxygen concentrations that are too low result in detrimental effects to the tissues as well as the entire body. Similarly, hypoxic conditioning of longer durations may have detrimental effects in addition to providing some desired beneficial effects. [Sharp, F., et al., Hypoxic Preconditioning Protects against Ischemic Brain Injury, NeuroRx: J. Am. Soc. Exp. Neuro., Vol. 1: 26-25 (2004)]

Initial understanding in the art about the effects of hypoxia and EPO focused on increased oxygenation of the blood via increased production of red blood cells. While increases in EPO production are believed to increase red blood cell production, its effects are not limited to this activity. Additional studies also show protective activity for EPO in the brain and heart as well as other organs. Furthermore, molecules such as HIF, induced by hypoxia, regulate EPO production in addition to a variety of other activities including metabolism, angiogenesis, and vascular tone—the stimulation of which may all play a role in protecting tissue from subsequent hypoxic or ischemic damage both prophylactically and post-ischemic events. [Eckardt K. U., Kurtz, A., Regulation of erythropoietin production, Eur. J. Clin. Invest., 35(Supp. 3):13-19, (2005)]. Further, hypoxia has also been shown to modulate glucose transporter proteins as well as improve glucose tolerance and insulin sensitivity. Modulation of glucose transporter proteins increases the ability of cell to regulate the amount of glucose in the blood via exchange of glucose between cells and the blood. [Chiu, L. L., et al., “Effect of Prolonged Intermittent Hypoxia and Exercise Training on Glucose Tolerance and Muscle GLUT4 Protein Expression in Rats”, J. Biomedical Sci., (2004), 11:838-846; Takagi, H., et al., “Hypoxia Upregulates Glucose Transport Activity Through an Adenosine-Mediated Increase of GLUT1 Expression in Retinal Capillary Endothelial Cells”, Diabetes, (1998) 47: 1480-1488.] In a separate study, hyperglycemia of diabetes was found to inhibit the activation of HIF-1α. The impaired ability to upregulate HIF-1α target genes has consequences for diabetes complications such as wound healing and retinopathy. This study further noted that administration of a known stimulator of HIF-1α aided in overcoming its hyperglycemic down-regulation often found in diabetic situations. [Catrina, S. B., et al., “Hyperglycemia Regulates Hypoxia-Inducible Factor-1α Protein Stability and Function”, Diabetes, (2004) 53: 3226-3232.]

It is also believed that the ability of CVAC therapy to provide increased blood flow, increased glucose transport, angiogenic and protective cellular responses, increased beta cell function, increased numbers of beta cells, EPO production, VEGF production, and HIF production can aid in recovery and repair of damaged tissues as well as facilitate treatment of diabetes and metabolic syndrome, including modulation of insulin production, insulin resistance, and glucose tolerance. Additionally, CVAC sessions are believed to act like a vaso-pneumatic pump on the user's body, thus stimulating flow of fluids in the body, including but not limited to blood and lymphatic fluids. The negative and positive pressures imposed by the CVAC session affect the fluid flow or movement within a body, thus improving the delivery of insulin, glucose, beneficial nutrients, immune factors, blood, and oxygen while also improving the removal of harmful toxins, fluids, and damaged cells or tissues. The combination of the beneficial effects of CVAC sessions results in improved regulation of insulin production and glucose tolerance.

In a number of retrospective studies, regular physical exercise has also appeared to be inversely related to the development of Alzheimer's. [Kiraly, M. A. and Kiraly, S. J., The effect of exercise on hippocampal integrity: review of recent research, Int. J. Psychiatry Med., 35(1): 75-89 (2005).] The Alzheimer's risk of those exercising regularly was reportedly half that of the least active. This research is consistent with the observation that virtually all measures designed to promote cardiac fitness and reduce stroke risk also seem to reduce Alzheimer's risk. [Kril, J. J. and Halliday, G. M., Alzheimer's disease: its diagnosis and pathogenesis, Int. Rev. Neurobiol., 48: 167-217 (2001).]

Traditional therapies for cancerous tumors involve chemotherapy, radiation or a combination of both, however neither addresses the problems associated with the hypoxic core of the tumor. Examples of tumors, although not limited to such examples, include mammary tumors (breast cancer), organ tumors (lung, colon, postate, liver, kidney, bladder, pancreas, etc.), brain tumors, testicular tumors, and ovarian tumors. Furthermore, both radiation and chemotherapy have known detrimental side-effects including destruction of prolific healthy tissues including, but not limited to, hair follicles, bone marrow, and stem cells. The compounds used for such treatments often face the problem of accessing the hypoxic core of a tumor that has reduced or cut off its blood supply. [Rosenberg, A. and Knox, S., Radiation sensitization with redox modulators: A promising approach, Int. J. Radiat. Oncol. Biol. Phys., 64(2):343-54 (2006).] However, alternative therapies such as hemoglobin supplementation, hematocrit augmentation, and oxygen deprivation are known to provide some beneficial effect. In the case of hemoglobin supplementation and hematocrit augmentation, chemical or biologic supplements are administered to patients while they undergo chemotherapy and/or radiation therapy. [Robinson, M. F., et al., Increased tumor oxygenation and radiation sensitivity in two rat tumors by a hemoglobin-based, oxygen-carrying preparation, Artif. Cells Blood Substit. Immobi. Biotechnol., 23(3): 431-8 (1995); Hirst, D. G., et al., The effect of alternations in haematocrit on tumour sensitivity to X-rays, In. J. Radiat. Biol. Relat. Stud. Phys. Chem. Med., 46(4):345-54 (1984).] The rise in hematocrit and/or hemoglobin due in part to EPO and related molecules also provides for increased oxygenation of tumor cores via increases in red blood cells as well as their oxygen-carrying capacity, yet effective treatment of cancerous tumors via static hypobaric conditioning remains somewhat unexplained [Herndon B. L. and Lally, J. J., Atmospheric pressure effects on tumor growth: hypobaric anoxia and growth of a murine transplantable tumor, J. Natl Cancer Inst., 70(4): 739-45 (1983)], and as noted above, excessive static hypobaric conditioning can result in detrimental effects and increases in hypoxia within cancerous tumors. [Vaupel P., et al., Impact of Hemoglobin Levels on Tumor Oxygenation: the Higher, the Better?, Strahlenther Onkol., 182(2):63-71 (2006).]

Current Treatments and Deficiencies of Treatments

Current treatments for cardiac and cerebral ischemia, and complications associated with or arising from such ischemias, encompass primarily behavioral changes, pharmaceutical therapies, and surgical intervention. Surgical intervention is quite traumatic to the body and can result in additional medical complications, especially where the body is already severely weakened or compromised due to the severity of the ischemia, the presence of congestive heart failure, and/or the over-all health and condition of the patient. Pharmaceuticals may also be used to treat ischemic attacks prophylactically or to aid in recovery. As with surgery, however, pharmaceuticals can bring on additional concerns due to negative side-effects from the compound itself, length of treatment, and unforeseen, individual reactions to the drugs. Furthermore, compliance with pharmaceutical regimens can be difficult over long term therapy.

Similarly, current methods or therapies for diabetes are few. Known traditional methods require the administration of pharmaceuticals through invasive routes and often require additional changes such as diet and exercise for which compliance is problematic. Thus, known diabetic treatments face many of the same obstacles described above for ischemia.

Current treatments for Alzheimer's disease are also few in number and at best marginally effective. Such treatments include various pharmaceuticals such as actylcholinesterase inhibitors as well as NSAIDs as mentioned above. Available non-pharmaceutical treatments include cardiovascular exercise, exercises for cognition and memory, as well as physical therapy for physical coordination and control. Furthermore, compliance with pharmaceutical regimes can be difficult as well as expensive, and compliance with non-pharmaceutical therapies can also be difficult to obtain with many patients well into the throes of the disease.

Current cancer treatments are detrimental to the healthy tissues of the body, and the length of treatments also contributes to the further destruction of healthy tissues. Many patients also become too weak for continued cancer treatments and are unable to successfully complete the treatments necessary to destroy the cancer.

While drugs and/or surgery can be used to treat many of the diseases or conditions described herein, there is a need for therapies which can be useful for treating or prevent such diseases and conditions without the associated physical trauma of surgery. There is a further need for therapies without the potential negative side-effects of pharmaceutical regimens. Alternatively, there is a need for such therapies that could lessen the negative side-effects of pharmaceutical regimens by altering pharmaceutical regimens, work beneficially with pharmaceutical regimens, or even work synergistically when used in combination with pharmaceutical regimens. There is a need for hypobaric or hypoxic conditioning which maximizes the beneficial effects within treatment periods that do not lead to the detrimental effects of such conditioning. There is a further need for such hypobaric or hypoxic conditioning that utilizes multiple and/or varying pressures throughout the conditioning. There is yet a further need for hypobaric or hypoxic conditioning that incorporates vaso-pneumatic considerations in addition to the hypoxic considerations.

CVAC provides exactly such an alternative. The methodology described herein provides for an application of hypobaric conditions for a variety of diseases and conditions that is superior to the current static hypobaric technologies. CVAC can be applied in myriad combinations, and in drastically reduced time-frames, as compared to the current hypobaric technologies. Prior hypobaric conditioning has focused on static conditions for relatively long treatment times. The invention and methodologies described herein provide a novel implementation and design of hypobaric technology as well as an advancement in its application.

### Methodology of the Cyclic Variations in Altitude Conditioning (CVAC) Program

A Pressure Vessel Unit (PVU) is a system for facilitating pressure changes accurately and quickly in the environment surrounding a user. A PVU can provide both reduced and increased atmospheric pressures. An example of a unique PVU and associated methods for controlling the pressure within such a PVU are described in U.S. Patent Publication number 2005/0056279 A1 and incorporated herein by reference. A variety of PVUs may be used in conjunction with the methods disclosed herein, including but not limited to those described in the U.S. Patent Publication number 2005/0056279, such as variable or fixed pressure and temperature hypobaric units. Other pressure units or chambers will be known to those of skill in the art and can be adapted for use with the disclosed methodologies.

The methodology of the present invention encompasses a set of pressure targets with defined transitions. Additional targets can be included such as temperature or humidity, and these targets can be implemented concurrently, prior to, or subsequent to the pressure targets. The permutations of targets are customizable to the individual and condition to be treated. Some of the terms relating to this methodology are defined below for a better understanding of the methodology as used in the context of the present invention.

A CVAC Program:

Every user will respond in a unique manner to changes in air pressure, temperature and oxygen levels that occur during cyclic variations in altitude conditioning. This necessitates a customized approach to delivering a highly effective and efficacious CVAC program to each user The program consists of a set of sessions, which are administered to the user as a serial round or cycle. This means that a user may have a session that they start and repeat a given number of times and then proceed to the next scheduled session which will be repeated a given number of times. A program may contain a set of one or more sessions, each of which preferably has a repetition schedule. The sessions are preferably delivered in a scheduled order, which repeats itself like a loop such that the user is administered one session at a time for a specified number of times. The user is then administered the next scheduled session a specified number of times. This process is preferably repeated until the user is administered the last element of the scheduled sessions set. When the requisite repetitions have been accomplished, preferably the process repeats itself beginning at the first element of the scheduled sessions set. A session or groups of sessions may be repeated multiple times before changing to a subsequent session or group of sessions, however, sessions may also be administered as few as one time before beginning the next session in the sequence. Subsequent sessions can contain targets that are identical to the previous session, or they can implement new permutations of desired targets. The combination of sessions and targets within sessions is customizable based on the desired physiological outcome and assessment of the user. Alternatively, a user may also modulate the parameters of a CVAC session, in certain embodiments from within the unit, thus providing for real-time user feedback and alterations. As used in reference to parameter of a CVAC session, modulation includes any changes, positive and negative, made to the parameters of the CVAC session. The parameters are described herein. This comprises a Cyclic Variations in Altitude Conditioning (CVAC) Program.

A CVAC Session:

A CVAC Session comprises of a set of targets which are multiple atmospheric pressures, and a CVAC session includes start and end points, and more than one target which is executed between the start and end points. These targets are delivered in a precise order that may vary and are executed in a variety of patterns including, but not limited to, cyclic, repeating, and/or linear variations. When a target is executed as contemplated herein, executed includes a change in pressure from one pressure value to another pressure value within a CVAC device as also described herein. The methodologies described herein are superior to previously described static hypobaric pressure therapies in multiple ways, which can include reduced time frames of application and unique variations and combinations of atmospheric pressures. Furthermore, CVAC sessions can also provide beneficial effects via the vas-pneumatic properties associated with the application of such sessions. The starting points and ending points in any CVAC session are preferably the ambient pressure at the delivery site. The targets inherent in any CVAC session are connected or joined together by defined transitions. These transitions are either increases in pressure (descent) or decreases in pressure (ascent), or a combination of the two. The nature of any transition may be characterized by the function of “delta P/T” (change in pressure over time). Transitions may be linear or produce a waveform. Preferably, all transitions produce a waveform. The most desirable waveforms are Sine, Trapezoidal and Square. Additional targets which modulate time, temperature, and/or humidity are also run concurrently, sequentially, or at other intervals with the pressure targets when such additional targets and conditions are desired. The entire collection of targets and transitions are preferably delivered in a twenty minute CVAC session, although the time of each session may vary in accordance with the desired outcome of the administration of the CVAC sessions. For example, CVAC sessions may be administered over minute increments such as 5, 10, 15, 16, 17, 18, 19, 20, 25, 30 minutes and/or more. The length of each CVAC session is customizable for each user.

A Set-Up Session:

The Set-Up Session may also be considered a Program. It is a single Session designed to prepare a new user for the more aggressive maneuvers or transitions encountered in the subsequent CVAC sessions that the user will undergo. The Set-Up session accounts for all ages and sizes and conditions, and assumes a minimal gradient per step exercise that allows the ear structures to be more pliant and to allow for more comfortable equalization of pressure in the ear structures. The purpose of the Set-Up session is to prepare a new user for their custom Program based upon the group into which they have been placed. The function of the Set-Up session is to qualify a user as being capable of adapting to multiple pressure changes in a given Session with acceptable or no discomfort. This is accomplished by instituting a gradient scale increase in pressure targets from very slight to larger increments with slow transitions increasing until a maximum transition from the widest difference in pressure targets is accomplished with no discomfort. Set-Up session transitions may be linear or produce a waveform. Preferably, all transitions are linear. The structure of a preferred Set-Up session is as follows: as with any session, the starting point and ending point is preferably at ambient pressure. A target equivalent to 1000 ft above ambient is accomplished via a smooth linear transit. A second target equivalent to 500 ft less than the first target is accomplished via a slow to moderate transit. These two steps are repeated until the user returns a “continue” or “pass” reply via an on-board interface. When the user has indicated that they are prepared to continue, the initial target (1000 ft) is increased by a factor of 500 ft, making it 1500 ft. The secondary target (500 ft less than the first target) remains the same throughout the session until the exit stage is reached. Each time the user indicates that they are ready to increase their gradient, the target is increased by a factor of 500 ft. At this time, the transits remain the same but the option of increasing gradient (shorter time factor) in the transits is available. A user preferably has the option of resuming a lower gradient if desired. There can be an appropriate icon or pad that allows for this option on the on-board interface display screen. Preferably, the Set-Up session lasts no longer than 20 minutes. A Set-Up session typically runs for twenty minutes maximum and executes a final descent to ambient atmospheric pressure upon beginning the last transit. The Set-Up session is a new user's Program until the user is able to fully complete the Set-Up session (that is to continue the targets and transits to the highest gradient) with no interrupts or aborts. When administering CVAC sessions for medical treatment, Set-Up sessions may be customized to suit the requirements of their medical condition. The determination of the appropriate Set-Up session can be made with guidance from or consultation with a user's qualified health professional, such as a treating physician.

The Interrupt:

During any phase in a session wherein a user desires to stop the session at that point for a short time, they may do so by activating an icon or other appropriate device on the on-board interface touch screen or control pad. This will hold the session at the stage of interruption for a predetermined time period, such as a minute, at which time the session will continue automatically. Preferably, a session may be interrupted three times after which a staged descent will occur and the user will be required to exit the pressure vessel. The user's file will be flagged and the user will be placed back on the Set-Up sessions until they can satisfactorily complete it. A warning or reminder may be displayed on the screen each time an interrupt is used that informs the user of how many times interrupt has been used and the consequences of further use. During any session, be it a Set-Up session or other type of session, a staged descent is also available if the user develops ear or sinus discomfort or wishes to terminate the session for any reason. A staged descent is characterized by slow, 1000 ft sine wave descent transits with re-ascensions of 500 ft at each step. The descents can be of greater or lesser transits but the ratio is usually about 1.5:1. At any time during the staged descent, the user can interrupt the descent and hold a given level or resume a previous level until comfort is achieved. The user may also re-ascend at their option if the staged descent is too aggressive. Any re-ascension is done in stages as described above. The user can subsequently indicate a “continue” on the descent and the staging will resume. This stepping continues until ambient pressure is reached whereupon the canopy opens such that the user can exit the pressure vessel.

The Abort:

When a user wishes to end a session immediately and quickly exit the pressure vessel, the abort function can be activated. Touching the “abort” icon on the on-board interface touch pad/screen enables this option. A secondary prompt is activated acknowledging the command and asking the user if they are sure they want to abort. The user indicates their commitment to the command by pressing “continue” or “yes”. The program is aborted and a linear moderate descent is accomplished to ambient pressure whereupon the canopy opens and the user exits. The user's file is flagged. The next time the user comes in for their session, the user is asked whether the abort was caused by discomfort. If yes, the user is placed back on the Set-Up session program. If no, the user is asked if they wish to resume their regularly scheduled session. The client is given the option of resuming their regularly scheduled CVAC session or returning to the Set-Up session.

### Program and Target Criteria, Including Medically Significant Criteria

Preferably, a user is categorized into a group of users having similar body-types with similar characteristics based upon answers to a questionnaire. The information from the questionnaire guides the construction of custom CVAC programs for each individual. When administering CVAC programs for cardiac or cerebral ischemia, diabetes or associated complications, Alzheimer's Disease, or cancer, the medical status of the user can also be used to determine appropriate pressures and additional parameters (such as duration, temperature, or humidity) of the targets. Custom session targets may be administered based upon the medical condition and therapy desired. The acceptable and appropriate target parameters may be obtained as described herein and through consultation with the user's physician or other appropriate health-care provider prior to designing session targets and administering a CVAC session. However the known contraindications of CVAC are similar to those of commercial air travel, allowing for a broad range of application.

### Methods of Treatment Using CVAC Sessions and the CVAC Program

CVAC sessions for the treatment of cardiac or cerebral ischemic disease, diabetes and associated complications, Alzheimer's disease, and cancer are administered preferably for at least 10 minutes, and more preferably at least 20 minutes, with variable frequency. Additional administration periods may include, but are not limited to, about 10 minutes, about 20 minutes, about 30 minutes, about 40 minutes, about 60 minutes, between 10 and 20 minutes, between 20 and 30 minutes, between 30 and 60 minutes, and between 60 and 120 minutes. Frequencies of sessions or series of sessions may include, but are not limited to, daily, monthly, or when medically indicated or prescribed. The frequency and duration of the sessions can be altered to suit the medical condition to be treated, and CVAC sessions may be administered as single sessions, or as a series of sessions, preferably with a Set-Up Session as described herein. For example, the frequency of sessions or series of sessions can be administered 3 times a week for 8 weeks, 4 times a week for 8 weeks, 5 times a week for 8 weeks, or 6 times a week for 8 weeks. Additional frequencies can be easily created for each individual user. Similarly, the targets in the sessions can also be altered or adjusted to suit the individual and medical condition to be treated. If at any time the user or attendant determines that the session is not being tolerated well, an abort may be initiated and the user brought down safely and exited. The permutations of targets can be customized to the individual, and may again be identified with the help of any person skilled in the art, such as a treating physician. Furthermore, the variations may be administered in regular intervals and sequence, as described, or in random intervals and sequence. The variations in number, frequency, and duration of targets and sessions can be applied to all methods of treatment with CVAC described herein.

Ischemia

In a first aspect of the invention, CVAC sessions are used to treat a wide variety of ischemia. As defined herein, treatment of ischemia includes prevention of ischemia, treatment of ischemia, prophylactic treatment of ischemia, amelioration of ischemia, as well as recovery from an ischemic event. In one embodiment of the present invention, at least one CVAC session is used to prophylactically treat users who are at risk for cerebral ischemia (strokes). A stroke is the acute neurological injury caused by any one of a variety of pathologic processes involving the blood vessels of the brain. Such processes may include occlusion of vessels, known weaknesses in vessel walls, inadequate cerebral flow, and rupture of cerebral vessels. Diagnosis of predisposal for stroke can be accomplished by any means commonly used in the medical community or by one of ordinary skill in the art.

In anticipation of a stroke, CVAC is administered to limit the injury to the brain or reduce the effects of ischemia. Treatment is administered through the use of one or more CVAC sessions. Such sessions may be user defined or custom-defined with input from the user's physician. A further embodiment of the invention includes the use of CVAC sessions when treatment for cerebral vessel occlusion or similar disease state is anticipated. CVAC sessions may be administered prior to such medical or surgical treatments to lessen the potential brain tissue injury that may occur. Many types of cardiac diseases, as well as arteriolosclerosis, may produce cerebral emboli. Intracardiac surgery, prosthetic valve replacement, heart bypass surgery, and angioplasty can all produce emboli which result in cerebral tissue damage. CVAC sessions may be administered in advance of any such surgeries or treatments to help reduce or prevent any damaging effects.

In another embodiment of the present invention, one or more CVAC sessions are used to ameliorate or prevent damage from ischemic heart disease. Ischemic heart disease relates to a broad spectrum of diseases caused by inadequate oxygen supply to the cardiac tissue. The oxygen deficiency may be caused by atherosclerotic obstruction of coronary arteries, non-atheromatous obstructions such as embolism, coronary artery spasm, hypertension or associated lifestyles which diminish the oxygen-carrying capacity of the blood such as smoking. Other lifestyle patterns known to influence cardiac disease are sedentary lifestyles, psychosocial tensions, and certain personality types or traits. An additional embodiment of the invention includes the use of CVAC sessions to reduce low density lipoproteins (LDL) in a user.

Administration of CVAC sessions prior to an actual cardiac ischemia can prophylactically treat the disease progression and complications associated with or arising from cardiac ischemia such as congestive heart failure. Prophylactic administration of CVAC sessions can also prevents or reduces the tissue damage in subsequent cardiac ischemic events. The ability of CVAC sessions to increase the blood flow, stimulate angiogenesis, and stimulate protective cellular responses conditions can condition tissues so that there is less necrotic damage during a subsequent cardiac ischemic event, allowing for quicker and more complete recovery from such events.

Similarly, CVAC sessions can be used to facilitate recovery following damage caused by ischemic heart disease as well as to treat congestive heart failure. Although not limited to a particular mechanism of action, it is believed that the ability of CVAC therapy to provide increased blood flow, increased red blood cell counts, angiogenic and protective cellular responses, EPO production, and HIF production can aid in recovery and repair of damaged tissues. When administered prophylactically, these same effects also condition tissues and prevent the detrimental effects of ischemia. Additionally, CVAC sessions are believed to act like a vaso-pneumatic pump on the user's body, thus stimulating flow of fluids in the body, including but not limited to, blood and lymphatic fluids. The negative and positive pressures imposed by the CVAC session affect the fluid flow or movement within a body, thus improving the delivery of beneficial nutrients, immune factors, blood, and oxygen while also improving the removal of harmful toxins, fluids, and damaged cells or tissues. The combination of the beneficial effects of CVAC sessions results in prevention, treatment, and improved recovery from heart disease, heart attacks, or other cardiac ischemic events.

Ischemic heart disease and cerebral ischemia are often asymptomatic until the extent of disease progression is well advanced. Preventative measures or therapies to control risk factors are often employed to address the asymptomatic situation. Typical preventative therapies include weight loss, change in diet, smoking cessation, physical exercise and conditioning, and stress reduction techniques. A physician or other person skilled in the art can identify and/or prescribe the aforementioned and additional preventative therapies. In one embodiment of the invention, one or more CVAC sessions are used in combination with these preventative, non-pharmaceutical measures to further aid in the prevention of, or reduction in damage from, subsequent cardiac and cerebral ischemic events. Combination treatments may be concurrent, sequential, or any other interval or frequency determined to be beneficial to the user.

Diabetes

Another aspect of the invention is the use of CVAC sessions for treatment of diabetes, including but not limited to uses to aid in regulation of insulin or insulin resistance and improving glucose tolerance as well as uses to treat or ameliorate complications associated with diabetes. Treatment of diabetes, as defined herein, includes, but is not limited to: treating metabolic syndrome, modulating insulin production, modulating insulin resistance, modulating glucose tolerance, and modulating glucose transport. In one embodiment of the present invention, Cyclic Variations in Altitude Conditioning Program is used to treat users who are in need of treatments for diabetes. Additional embodiments include the administration of CVAC to modulate insulin production, modulate glucose tolerance, increase the oxygenation of the blood, increase the number of red blood cells within a user, increase angiogenesis and improve transport of glucose and insulin, increase the production of HIF's, upregulate the glucose transport system, and/or stimulate other associated physiological processes affected by CVAC treatment such as fluid (lymph, blood, or other bodily fluids) movement. Treatment is administered through the use of one or more CVAC sessions. Such sessions may be user defined or custom-defined with input from the user's physician. CVAC sessions may be administered in advance of any standard diabetes therapies, preferably more productively and efficiently than standard therapies, reduce the need for standard therapies, preferably more efficiently than standard therapies, and facilitate insulin production and glucose tolerance, preferably faster and more efficiently than standard therapies.

Additionally, CVAC is not limited to application with Type 2 Diabetes, and CVAC sessions may be administered in a similar manner to any type of diabetes therapy involving the regulation of insulin, glucose tolerance, and glucose transport. Similarly, CVAC therapy can be utilized to prevent, treat, or ameliorate metabolic syndrome. Further embodiments of the invention include application of CVAC for the treatment of complications associate with and/or arising from diabetes. Complications such as visual disorders, vascular diseases, and kidney diseases may be treated with CVAC sessions. The aforementioned mechanisms of action attributable to CVAC may all contribute to the treatment and/or amelioration of diabetic complications. Modulation of angiogenesis, fluid and blood production, insulin and glucose tolerance, molecular factors such as HIF-1α and related hypoxia-induced genes as well as the vaso-pneumatic effects may benefit the known complications associated with and/or arising from diabetes as well as treating the underlying diabetes.

One embodiment includes the treatment of vascular diseases associated with diabetes such as lower extremity ulceration and amputation. CVAC is administered to modulate insulin production, modulate glucose tolerance, increase the oxygenation of the blood, increase the number of red blood cells within a user, increase angiogenesis and improve transport of glucose and insulin, increase the production of HIF's, upregulate the glucose transport system, and/or stimulate other associated physiological processes affected by CVAC treatment such as fluid (lymph, blood, or other bodily fluids) movement. As in the treatment of diabetes, these mechanisms of action, but not limited to only these, are believed to ameliorate or modulate healing of any complications associated with and/or arising from diabetes including diabetic ulcers, bodily fluid flow such as blood and lymph, angiogenesis and protective cellular responses, hypertension and associate heart disease, vision disorders such as glaucoma and retinopathy, and kidney diseases.

An additional embodiment of the invention disclosed herein includes the treatment of metabolic syndrome. CVAC sessions are administered to facilitate the treatment, prevention, and/or amelioration of metabolic syndrome. As with the aforementioned embodiments, the application of CVAC sessions can modulate a variety of physiological parameters associated with metabolic syndrome, including insulin resistance, glucose tolerance, and glucose transport.

Alzheimer's Disease

In another aspect of the present invention, CVAC is used to treat users who have Alzheimer's disease, symptoms of the disease, or who exhibit risk factors associated with increased risk of Alzheimer's disease such as diabetes, hypertension, high cholesterol, and smoking. CVAC is administered to increase the oxygenation of the affected tissue (e.g. the brain), increase the production of HIFs, and/or stimulate other associated physiological processes affected by CVAC treatment such as fluid (lymph, blood, cerebral, spinal, or other bodily fluids) movement. Treatment is administered through the use of one or more CVAC sessions. Such sessions may be user defined or custom-defined with input from the user's physician. CVAC sessions may be administered in advance of other treatment regimens to help reduce or prevent any damaging effects.

Although not limited to a particular mechanism of action, it is believed that the ability of CVAC therapy to provide increased blood flow, increased red blood cell counts, angiogenic and protective cellular responses, EPO production, VEGF production, and HIF production aid sin recovery and repair of damaged tissues and can also prevent the onset or progression of Alzheimer's disease. Further, CVAC's vaso-pneumatic pump action stimulates flow of fluids in the body, including but not limited to blood, lymphatic, cerebral, and spinal fluids. The negative and positive pressures imposed by the CVAC session affect the fluid flow or movement within a body, thus improving the delivery of beneficial nutrients, immune factors, blood, and oxygen while also improving the removal of harmful toxins, fluids, and damaged cells or tissues. Again, the combination of the beneficial effects of CVAC sessions results in the treatment of Alzheimer's disease such as prevention of the onset of the disease and retardation of disease progression.

Cancer

In an additional aspect of the present invention, CVAC sessions are used to treat users who are suffering from cancer, cancerous tumors, and/or combinations thereof Examples of tumors, although not limited to such examples, include mammary tumors (breast cancer), organ tumors (lung, colon, postate, liver, kidney, bladder, pancreas, etc.), brain tumors, testicular tumors, and ovarian tumors. In one embodiment, CVAC is administered to increase the oxygenation of and provide treatment to the cancerous tissue, increase the production of HIF's, and stimulate other associated physiological processes affected by CVAC treatment such as fluid (lymph, blood, or other bodily fluids) movement. Treatment is administered through the use of one or more CVAC sessions. Such sessions may be user defined or custom-defined with input from the user's physician. CVAC sessions may be administered in advance of, during, or following other treatment regimens to improve the efficacy of such treatments and/or reduce or prevent any damaging effects from such treatments. In an additional embodiment of the present invention, CVAC is used to help users better tolerate initial or subsequent administration of cancer therapies such as chemotherapy, radiation therapy, and combinations thereof. Similarly, CVAC is used to help users better tolerate subsequent administration of more severe and/or multiple chemotherapy sessions, radiation sessions, or combinations thereof.

Symptomatic individuals are often placed on a pharmaceutical regimen to treat their ischemic disease state, diabetes, Alzheimer's or cancer. CVAC sessions may also be used in combination with pharmaceutical regimens to prevent, treat, or ameliorate such diseases and conditions. CVAC sessions may also be used in combination with pharmaceutical regimens or non-pharmaceutical therapies such as physical therapy to treat, ameliorate or prevent further aforementioned damage or disease progression. In all the aforementioned aspects and embodiments, CVAC sessions of any combination or permutation can be administered prior to, concurrent with, or subsequent to administration of a pharmaceutical or pharmaceuticals. Multiple permutations of pharmaceutical and CVAC session combinations are possible, and combinations appropriate for the type of medical condition and specific pharmaceutical may be identified with the help of any person skilled in the art, such as a treating physician.

Although not limited, it is believed that the ability of CVAC therapy to provide increased blood flow, increased red blood cell counts, angiogenic and protective cellular responses, EPO production, and HIF production can aid in recovery and repair of damaged tissues. When administered prophylactically, these same effects also condition tissues and prevent the detrimental effects of ischemia, diabetes, Alzheimer's disease, and/or cancer. Additionally, CVAC sessions are believed to act like a vaso-pneumatic pump on the user's body, thus stimulating flow of fluids in the body, including but not limited to blood and lymphatic fluids. The negative and positive pressures imposed by the CVAC session affect the fluid flow or movement within a body, thus improving the delivery of beneficial nutrients, immune factors, blood, and oxygen while also improving the removal of harmful toxins, fluids, and damaged cells or tissues. The combination of the beneficial effects of CVAC sessions results in prevention, improved treatment, and improved recovery from strokes or other cerebral ischemic events, diabetes and associated complications, Alzheimer's disease, and cancer.

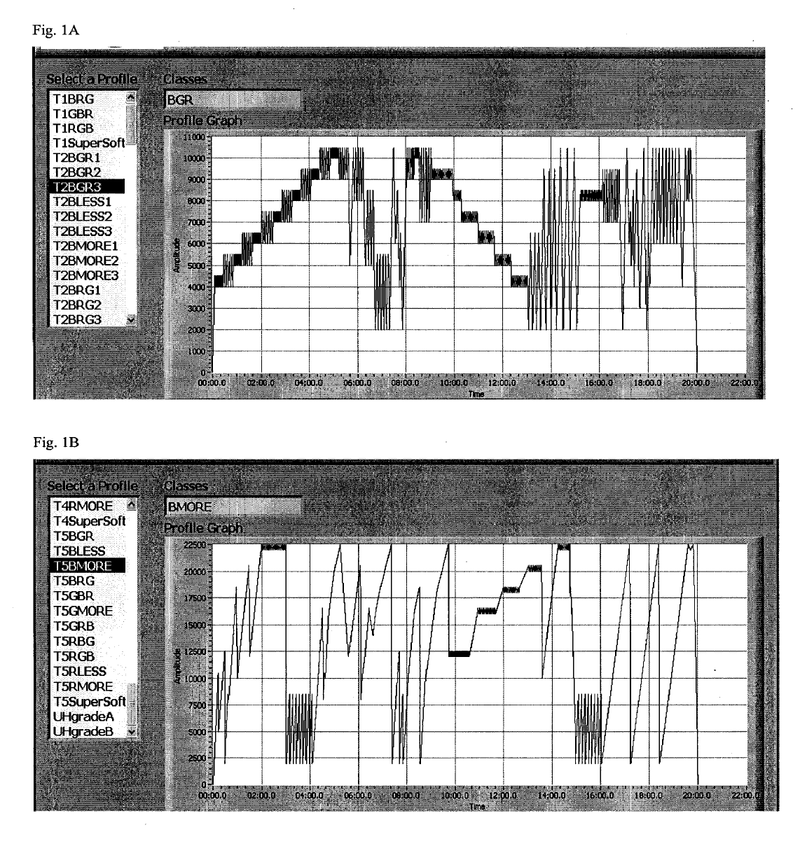

Specific examples of a CVAC session are shown graphically in FIGS. 1A and 1B. In both figures, the parameters of the program are shown as a line graph with axes that correspond to time (x-axis) and pressure change (y-axis).

Efficacy of CVAC Treatments

Ischemia

Efficacy of CVAC treatments for cardiac and cerebral ischemia can be evaluated with a variety of imaging and assessment techniques known in the art. Examples include methods such as magnetic resonance imaging (MRI) of the affected region, invasive imaging through catheterization, or alternative non-invasive imaging methods. Additional assessment criteria known in the art include: hematocrit measurement, blood-gas analysis, extent of blood-perfusion of tissues, angiogenesis within tissues, erythropoietin production, extent of tissue necropsy following ischemic events, and assessment of cognitive abilities and/or motor skills following ischemic events.

By example only, when hematocrit is the physiological marker used to assess CVAC efficacy, modulation of hematocrit during or following one or more CVAC sessions is indicative of efficacious CVAC treatment for the treatment, amelioration, or prevention of ischemic events. In one embodiment, an increase in hematocrit is indicative of efficacious CVAC treatment. Conversely, a lack of change in the user's hematocrit (or with any of the physiological markers described herein) does not necessarily indicate that the CVAC treatments are not achieving positive results. Similarly, when blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy. When blood-perfusion of the tissues is the physiological marker used to assess CVAC efficacy, increases in blood volumes and/or blood exchange within tissues during or following one or more CVAC sessions are indicative of the efficacious CVAC treatment. Angiogenesis within affected tissues can also be a physiological marker used to assess CVAC efficacy. Modulation of vessel development within the affected tissues during or following one or more CVAC sessions is indicative of efficacious CVAC treatments. Additionally, initiation or modulation of VEGF expression within affected tissues during or following one or more CVAC sessions is also indicative of efficacious CVAC treatment. Modulation of erythropoietin production following one or more CVAC sessions is also a physiological marker used to assess the efficacy of CVAC treatments. In one embodiment of the present invention, increases in the expression of erythropoietin indicate efficacious CVAC treatments. Extent of tissue necropsy is a further physiological marker used to assess CVAC efficacy. Modulation of tissue necropsy, including repair and/or efficient removal of affected tissue by known bodily repair systems, pathways, and cascades as well as prevention of initial or continued necrosis, during or following one or more CVAC sessions is indicative of CVAC session efficacy. Still further physiological markers for assessing efficacy of CVAC sessions include modulation of cognitive and/or motor skills during or following one or more CVAC sessions. In one embodiment, improved or increased motor skills are indicative of efficacious CVAC treatment. Similarly, in yet another embodiment improved cognitive skills are indicative of efficacious CVAC treatment. Assessment of CVAC efficacy in treating congestive heart failure may include all aforementioned techniques and criteria. In addition, efficacy of CVAC session for the treatment, prevention, and/or amelioration of congestive heart failure may be assessed by monitoring swelling or fluid collection in body tissues. In one embodiment, the reduction of swelling in the legs and ankles following the administration of one or more CVAC sessions is indicative of efficacious treatment. Additional criteria for assessing the treatment and prevention of ischemic damage or ischemic events will be known by those of skill in the art and can be employed to assess the beneficial effects of CVAC programs.

Diabetes and Associated Complications

Efficacy of CVAC treatments for modulation of insulin regulation, glucose tolerance, and glucose transport can be evaluated with a variety of imaging and assessment techniques known in the art. Assessment criteria known in the art include, but are not limited to: assessment of insulin levels, assessment of blood glucose levels and glucose uptake studies by oral glucose challenge, assessment of cytokine profiles, blood-gas analysis, extent of blood-perfusion of tissues, and angiogenesis within tissues. Additional criteria for assessing the treatment of diabetes will be known by those of skill in the art and can be employed to assess the beneficial effects of CVAC programs.

By example only, modulation of insulin levels is indicative of efficacious CVAC treatments. Conversely, a lack of change in the user's insulin (or with any of the physiological markers described herein) does not necessarily indicate that the CVAC treatments are not achieving positive results. Modulation of insulin resistance is also indicative of efficacious CVAC treatments. Similarly, modulation of glucose levels is indicative of efficacious CVAC treatment, and modulation of glucose transport is indicative of CVAC efficacy for diabetes therapy. Glucose transport may be monitored by, although not limited to, examination of GLUT protein expression (any of the genes defined as falling within the GLUT family) and/or glut gene expression. Angiogenesis within affected tissues can also be a physiological marker used to assess CVAC efficacy. Modulation of vessel development within the tissues or body of a user during or following one or more CVAC sessions is indicative of efficacious CVAC treatments. Again, by example only, angiogenesis may be assessed by a variety of imaging and detection methods including dyes, MRI, fluoroscopy, endoscopy, and other means known in the art. Additionally, initiation or modulation of VEGF expression within affected tissues during or following one or more CVAC sessions is also indicative of efficacious CVAC treatment. Modulation of EPO production following one or more CVAC sessions is also a physiological marker used to assess the efficacy of CVAC treatments. In one embodiment of the present invention, increases in the expression of EPO indicate efficacious CVAC treatments. Similarly, when blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy.

In one embodiment, an increase in insulin production following at least one CVAC treatment (as compared with measurements taken pre-CVAC treatment) is indicative of a positive effect of the CVAC treatment on the function of beta cells and production of insulin. In a further embodiment, modulation of HbA1c is indicative of efficacious CVAC treatment. HbA1c is a known protein found in the blood, whose levels are representative of blood glucose levels. In yet another embodiment, a positive result following administration of an oral glucose challenge test (as compared with results of an oral glucose challenge test administered pre-CVAC treatment) is indicative of a positive effect on the body's glucose tolerance from the CVAC treatment. The administration of such tests and measurements will be well known to those of skill in the art.

Efficacy of CVAC treatments for the modulation, treatment, and/or amelioration of complications of diabetes may be assessed by a variety of techniques known in the art. For example, efficacy of CVAC for healing of diabetic ulceration may assessed by extent of healing of the ulceration or change in healing time of the ulceration during or following administration of one or more CVAC sessions. Similarly, prevention of ulceration may be assessed by analysis of ulceration incidence within a CVAC treated population relative to a control population. Modulation of angiogenesis during or following one or more CVAC sessions may be indicative of CVAC efficacy for the amelioration and/or treatment of vascular diseases in diabetic patients. Modulation of urinary albumin excretion during or following one or more CVAC sessions may be indicative of CVAC efficacy for the treatment or amelioration of kidney or renal disease in diabetic patients. Additional criteria for assessing the treatment of diabetes complications will be known by those of skill in the art and can be employed to assess the beneficial effects of CVAC programs for such indications.

Alzheimer's Disease

Efficacy of CVAC treatments for Alzheimer's disease can be evaluated with a variety of imaging and assessment techniques known in the art. Examples include methods such as magnetic resonance imaging (MRI) of the affected region, invasive imaging through catheterization, or alternative non-invasive imaging methods. Additional assessment criteria known in the art include: hematocrit measurement, blood-gas analysis, extent of blood-perfusion of tissues, angiogenesis within tissues, erythropoietin production, extent of plaque formation in the affected tissues, and assessment of additional indicators such as speech and cognitive ability, memory and recognition, as well as physical coordination and movement. Additional criteria for assessing the treatment of Alzheimer's disease will be known by those of skill in the art and can be employed to assess the beneficial effects of CVAC programs.

By example only, extent of amyloid plaque formation is a physiological marker used to assess CVAC efficacy. Modulation of amyloid plaque formation including repair or efficient removal of affected tissue by known bodily repair systems, pathways, and cascades as well as prevention of initial or continued plaque formation, during or following one or more CVAC sessions is indicative of CVAC session efficacy. Conversely, a lack of change in the user's amyloid plaque formation (or with any of the physiological markers described herein) does not necessarily indicate that the CVAC treatments are not achieving positive results. Additional assessment criteria for the efficacy of CVAC sessions include modulation of cognitive skills, memory capability, recognition skills, physical coordination and movement skill, and combinations thereof during or following one or more CVAC sessions. In yet another embodiment, modulation of immune or inflammation-mediating cells present in the affected tissue, chemokine and cytokine profiles in the affected tissue, or other immune-cell factors or combinations thereof is also indicative of efficacious CVAC treatment. For example, cytokine profiles of interleukins within the affected tissues or body can be monitored to determine efficacy of CVAC treatments. Angiogenesis within affected tissues can also be a physiological marker used to assess CVAC efficacy. Modulation of vessel development within the affected tissues during or following one or more CVAC sessions is indicative of efficacious CVAC treatments. Again, by example only, angiogenesis may be assessed by a variety of imaging and detection methods including dyes, x-ray, MRI, fluoroscopy, endoscopy, and other means known in the art. Additionally, initiation or modulation of VEGF expression within affected tissues during or following one or more CVAC sessions is also indicative of efficacious CVAC treatment. Modulation of erythropoietin production following one or more CVAC sessions is also a physiological marker used to assess the efficacy of CVAC treatments. In one embodiment of the present invention, increases in the expression or amount of circulating erythropoietin indicate efficacious CVAC treatments. Further, when hematocrit is the physiological marker used to assess CVAC efficacy, modulation of hematocrit during or following one or more CVAC sessions is indicative of efficacious CVAC treatment for the treatment of Alzheimer's disease. In one embodiment, an increase in hematocrit is indicative of efficacious CVAC treatment. Similarly, when blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy. When blood-perfusion of the tissues is the physiological marker used to assess CVAC efficacy, increases in blood volumes or blood exchange and combinations thereof within tissues during or following one or more CVAC sessions are indicative of the efficacious CVAC treatment. Additional criteria for assessing the treatment of Alzheimer's disease will be known by those of skill in the art and can be employed to assess the beneficial effects of CVAC programs.

Cancer

Efficacy of CVAC treatments for cancer can be evaluated with a variety of imaging and assessment techniques known in the art. Examples include methods such as magnetic resonance imaging (MRI) of the affected region, invasive imaging through catheterization, or alternative non-invasive imaging methods. Additional assessment criteria useful in assessing the efficacy of CVAC sessions for treatment of cancer include: hematocrit measurement, blood-gas analysis, extent of blood-perfusion of tissues, angiogenesis within tissues, erythropoietin production, extent of tissue necropsy in the affected tissues, and assessment of additional physical indicators such as reduction in tumor or cancerous tissue size and/or reduction in the number of metastases. Assessment of immune or inflammation-mediating cells present in the affect tissue, chemokine and cytokine profiles in the affected tissue, or other immune-cell factors can also aid in the evaluation of efficacy. Additional criteria for assessing the treatment cancer will be known by those of skill in the art and can be employed to assess the initial or further beneficial effects of CVAC programs.

By example only, modulation of erythropoietin production following one or more CVAC sessions is a physiological marker used to assess the efficacy of CVAC treatments. For example, but not limited to, increases in the expression of erythropoietin indicate efficacious CVAC treatments. Conversely, a lack of change in the user's erythropoietin levels (or with any of the physiological markers described herein) does not necessarily indicate that the CVAC treatments are not achieving positive results. In another embodiment, an increase in hematocrit is indicative of efficacious CVAC treatment. When hematocrit is the physiological marker used to assess CVAC efficacy, modulation of hematocrit during or following one or more CVAC sessions is indicative of efficacious CVAC treatment for the treatment of cancer. Extent of tissue necropsy is a further physiological marker used to assess CVAC efficacy. Modulation of tissue necropsy, including repair or efficient removal of affected tissue by known bodily repair systems, pathways, and cascades during or following one or more CVAC sessions is indicative of CVAC session efficacy. Still further physical indicators for assessing efficacy of CVAC sessions include modulation of cancerous tissue or tumor size and/or combinations thereof during or following one or more CVAC sessions. In one embodiment, reduced size of cancerous tissue masses and/or tumor masses are indicative of efficacious CVAC treatment. In a further embodiment, a reduction or prevention of metastases within a user's body is indicative of CVAC efficacy. Similarly, reduction of cancerous tissue in the body via detection of cancerous tissue antigens with suitable detection antibodies, molecules, and/or compounds can also be used to assess the efficacy of CVAC sessions for cancer treatment. Further embodiments include blood-gas analysis. When blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy. When blood-perfusion of the tissues is the physiological marker used to assess CVAC efficacy, increases in blood volumes or blood exchange and combinations thereof within tissues during or following one or more CVAC sessions are indicative of the efficacious CVAC treatment. Angiogenesis within affected tissues can also be a physiological marker used to assess CVAC efficacy. Modulation of vessel development within the affected tissues during or following one or more CVAC sessions is indicative of efficacious CVAC treatments. Additionally, initiation or modulation of VEGF expression within affected tissues during or following one or more CVAC sessions is also indicative of efficacious CVAC treatment. Similarly, in yet another embodiment modulation of immune or inflammation-mediating cells present in the affected tissue, antibodies to cancerous tissue or tumor antigens, chemokine and cytokine profiles in the affected tissue, or other immune-cell factors or a combination thereof is also indicative of efficacious CVAC treatment. For example, cytokine profiles of interleukins within the affected tissues or body can be monitored to determine efficacy of CVAC treatments. Additional criteria for assessing the treatment of cancer will be known by those of skill in the art and can be employed to assess the beneficial effects of CVAC programs.

A method for treating ischemic disease, diabetes and complications associated therewith, Alzheimer's disease, and cancer by administration of various environmental pressure levels for hypoxic conditioning is disclosed herein. Previously described PVU and CVAC methodology is used to implement the methods, and alternative PVUs can be used with the disclosed methodologies.

### EXAMPLES

### Example 1

To assess the efficacy of CVAC sessions, four individuals were administered CVAC sessions and their red blood cell counts hematocrit were subsequently measured and the levels recorded. Increases in red blood cell counts are indicative of CVAC session efficacy, and changes in hematocrit similarly indicate changes in erythropoiesis. For the study, CVAC sessions were administered to a group of four individuals for 40 minutes, 4 times a week, over an 8 week period. Red blood cell levels (RBC) were measured at 5 different intervals during the 8 week test period. The results of the study were as follows:

RBC mean increase: 4.7%

The increases in RBC's indicate that CVAC sessions were successful in positively modulating red blood cell counts as well as hematocrit, and both measurements are indicative of increased erythropoiesis. Thus, the administration of CVAC sessions successfully improved erythropoiesis in this 8 week study.

### Example 2

In the same study as example 1, to assess the efficacy of CVAC sessions four individuals were administered CVAC sessions and their hematocrit was subsequently measured and the levels recorded. Changes in hematocrit indicate changes red blood cell concentration as well as indicating changes in erythropoiesis. For the study, CVAC sessions were administered to a group of four individuals for 40 minutes, 4 times a week, over an 8 week period. Hematocrit (HCT) was measured at 5 different intervals during the 8 week test period. The results of the study were as follows:

HCT mean increase: 5.3%

The increases in HCT, both alone in combination with the RBC increase as described in example 1, indicate that CVAC sessions were successful in positively modulating hematocrit levels and are further indicative of increased erythropoiesis. Thus, the administration of CVAC sessions successfully improved erythropoiesis in this 8 week study.

### Example 3

To assess the efficacy of CVAC sessions, 13 individuals, all between the ages of 20 and 40 years old, were administered CVAC sessions and changes in their erythropoietin (EPO) levels were measured. Frequency of CVAC administration was for one hour per day, 5 days per week, for seven weeks. Increases in EPO were measured prior to administration of CVAC and three hours post-administration of CVAC, and EPO concentration is expressed as mIU/ml. Thus changes in EPO can be represented by the formula: deltaEPO=Post-CVAC EPO mIU/ml−pre-CVAC EPO mIU/ml. The study found that EPO levels changed significantly over the study period in the population. Specifically, mean changes in EPO concentration increased from 0.2 mIU/ml following the first 2 weeks of CVAC administration to 2.0 mIU/ml following 8 weeks of the CVAC administration. The significant changes in EPO levels found in the study population indicate that the administration of CVAC sessions can positively modulate EPO production, hence providing an alternative and efficacious method to exogenous EPO administration.

### Example 4

Two diabetic subjects (Type-1 and Type-2) were administered 20 minute CVAC sessions, three times a week over a 9 week period. Triglicerides (TGC), Cholesterol levels (HDL and LDL), and Hemoglobin A1c levels were assessed at time points during the study period. Study time periods and results were as follows:

| | Baseline | | 4 Weeks | | 9 Weeks | |
|---|---|---|---|---|---|---|
| Physiological | Subject | Subject | Subject | Subject | Subject | Subject |
| Marker | #1 | #2 | #1 | #2 | #1 | #2 |
| Triglycerides | 102 | 81 | 118 | 85 | 101 | n/d |
| (TGC) | | | | | | |
| HDL | 49 | 72 | 49 | 76 | 49 | n/d |
| LDL | 106 | 111 | 67 | 99 | 84 | n/d |
| HbA1c | 6.7 | 8.4 | 6.8 | 7.6 | 7.1 | n/d |
| (LDL + TGC)/ | 4.2 | 2.7 | 3.8 | 2.4 | 2.1 | n/d |
| HDL | | | | | | |

Subject #1: Type-2 diabetic, female
Subject #2: Type-1 diabetic, male

The results from the two different subjects show a significant drop in their (LDL+TGC)/HDL ratios, indicating improvement in HDL as well as reductions in LDL and/or TGC. Thus in this study, the administration of CVAC sessions resulted in a greater than 9% reduction in the (LDL+TGC)/HDL ratio, successfully reduced the LDL and TGC levels of diabetic individuals, and raised the HDL levels in the diabetic individuals. It may additionally result in at least a 5% reduction in the (LDL+TGC)/HDL ratio, at least a 5-10% reduction in the (LDL+TGC)/HDL ratio, or greater than a 10% reduction in the (LDL+TGC)/HDL ration.

The aspects and embodiments of the present invention described above are only examples and are not limiting in any way. Various changes, modifications or alternations to these embodiments may be made without departing from the spirit of the invention and the scope of the claims.

What is claimed is:

1. A method of treating ischemia in a mammal comprising the step of administering at least one CVAC session, said CVAC session having a start point, an end point and more than one target which is executed between said start point and said end point.

2. The method of claim 1, wherein said CVAC session is administered to treat cerebral ischemia.

3. The method of claim 1, wherein said CVAC session is administered to treat ischemic heart disease.

4. The method of claim 1, wherein said CVAC session is administered to reduce LDL.

5. A method of treating congestive heart failure in a mammal comprising the step of administering at least one CVAC session, said CVAC session having a start point, an end point and more than one target which is executed between said start point and said end point.

6. The method of claim 1, further comprising the step of measuring efficacy of CVAC sessions via changes in physiological markers.

7. The method of claim 6, wherein the physiological marker measured is selected from among:
hematocrit;
erythropoietin (EPO) production;
blood gas composition;
oxygenation of tissues;
angiogenesis within tissues;
blood-perfusion of tissues;
or extent of tissue necropsy following an ischemic event.

8. The method of claim 1, further comprising the step of administering least one pharmaceutical compound.

9. The method of claim 1 wherein the user can modulate the parameters of a session.

10. A method of treating diabetes in a mammal comprising the step of administering at least one CVAC session, said CVAC session having a start point, an end point and more than one target which is executed between said start point and said end point.

11. A method of treating one or more complications associated with or arising from diabetes in a mammal comprising the step of administering at least one CVAC session, said CVAC session having a start point, an end point and more than one target which is executed between said start point and said end point, said one or more complications selected from the group consisting of: diabetic ulcerations, vascular disease, heart disease, visual disorders, kidney disease, and combinations thereof.

12. The method of claim 10, further comprising the step of measuring efficacy of the at least one CVAC session via changes in physiological markers.

13. The method of claim 12, wherein the physiological marker is selected from among:
insulin;
HbA1c;
glucose tolerance;
glucose transport;
GLUT expression;
HIF-1α expression;
VEGF production;
hematocrit;
erythropoietin production;
oxygenation of tissues in the mammal;
or angiogenesis within tissues of the mammal.

14. The method of claim 10, further comprising the step of administering least one pharmaceutical compound.

15. The method of claims 14, wherein the at least one pharmaceutical compound is insulin.

16. A method of treating Alzheimer's disease in a mammal comprising the step of administering at least one CVAC session, said CVAC session having a start point, an end point and more than one target which is executed between said start point and said end point.

17. The method of claim 16, further comprising the step of measuring efficacy of the at least one CVAC session via changes in assessment criteria.

18. The method of claim 17, wherein the assessment criterion is selected from among:
extent of plaque formation;
modulation of cognitive skills;
modulation of memory skills;
modulation of recognition skills;
modulation of physical coordination;
angiogenesis;
blood-perfusion of tissues;
VEGF production;
hematocrit;
erythropoietin (EPO) production;
blood gas composition;
or oxygenation of tissues.

19. A method of treating cancer in a mammal comprising the step of administering at least one CVAC session, said CVAC session having a start point, an end point and more than one target which is executed between said start point and said end point.

20. The method of claim 19 wherein the cancer treated is a tumor.

21. The method of claim 19, wherein said treatment comprises the additional step of administering a chemotherapeutic agent.

22. The method of claim 19, wherein said treatment comprises the additional step of administering radiation.

23. The method of claim 19, further comprising the step of administering least one pharmaceutical compound.

24. The method of claim 19, wherein the user can modulate the parameters of a session.

25. The method of claim 19, further comprising the step of measuring efficacy of CVAC sessions via changes in physiological markers.

26. The method of claim 25, wherein the physiological marker is selected from among:
extent of tissue necropsy;
cancerous tissue size;
number of metastases of cancerous tissue;
hematocrit;
erythropoietin (EPO) production;
blood gas composition;
oxygenation of tissues;
angiogenesis within tissues;
or blood-perfusion of tissues.
